(19) United States
(12) Patent Application Publication
(10) Pub. No.: US 2025/0040955 A1
(43) Pub. Date: Feb. 6, 2025

(54) Tissue-Removing Catheter with Flexible and Stiff Portions

(71) Applicant: MEDTRONIC VASCULAR INC., Santa Rosa, CA (US)

(72) Inventors: Aran Murray, Galway (IE); Ronan M. Finn, Galway (IE); Aram Jamous, Athenry (IE); Alan Ryan, Moycullen (IE); Cian Walsh, Oranmore (IE); Caoimhe Marie Reilly, Castlegar (IE); Martin Bernard Patten, Westport (IE); Meriam Zahraa Waad Jassim, Limerick (IE); Kevin Bancud, Doughiska (IE); Thomas P. Hayden, Turloughmore (IE); Conor J. Casey, Oranmore (IE)

(21) Appl. No.: 18/714,946

(22) PCT Filed: Nov. 30, 2022
(86) PCT No.: PCT/US2022/051390

§ 371 Date: May 30, 2024

Related U.S. Application Data
(60) Provisional application No. 63/284,387, filed on Nov. 30, 2021.

(51) Int. Cl. A61B 17/3207; A61B 17/00

(52) U.S. Cl. CPC A61B 17/320758; A61B 17/00234; A61B 2017/00238; A61B 2017/00305; A61B 2017/00398; A61B 2017/00477

(57) ABSTRACT

The present disclosure provides a tissue-removing catheter for removing tissue in a body lumen. The tissue-removing catheter includes an elongate drive member and a tissue-removing element. The elongate drive member has proximal and distal ends with a length and a longitudinal axis extending between the proximal and distal ends. The elongate drive member is sized and shaped to be received in the body lumen and rotates about its longitudinal axis. The elongate drive member further includes first and second longitudinal portions. The first longitudinal portion is proximal to and stiffer than the second longitudinal portion. The tissue-removing element is operatively coupled to the distal end of the elongate drive member and rotates by the elongate drive member to remove the tissue in the body lumen.

### FIELD

The present disclosure generally relates to a tissue-removing catheter.

### BACKGROUND

Tissue-removing catheters are used to remove unwanted tissue in body lumens. As an example, atherectomy catheters are used to remove material from a blood vessel to open the blood vessel and improve blood flow through the vessel. This process can be used to prepare lesions within a patient's coronary artery to facilitate percutaneous coronary angioplasty (PTCA) or stent delivery in patients with severely calcified coronary artery lesions. Atherectomy catheters typically employ a rotating element which is used to abrade or otherwise break up the unwanted tissue.

### SUMMARY

In one aspect, the present disclosure provides a tissue-removing catheter for removing tissue in a body lumen. The tissue-removing catheter includes an elongate drive member and a tissue-removing element. The elongate drive member has proximal and distal ends with a length and a longitudinal axis extending between the proximal and distal ends. The elongate drive member is sized and shaped to be received in the body lumen and rotates about its longitudinal axis. The elongate drive member further includes first and second longitudinal portions. The first longitudinal portion is proximal to and stiffer than the second longitudinal portion. The tissue-removing element is operatively coupled to the distal end of the elongate drive member and rotates by the elongate drive member to remove the tissue in the body lumen.

### BRIEF DESCRIPTION OF THE DRAWINGS

FIG. 1 is an illustration of an example of a catheter of the present disclosure including a coupler;

FIG. 2 is an enlarged view of a catheter body of the catheter in FIG. 1.

FIG. 3 is an illustration of another example the catheter including an alternative coupler.

FIG. 4 is an enlarged view of a catheter body of the catheter in FIG. 3.

FIG. 5 is an illustration of an additional example of the catheter including a laminate.

FIG. 6 is an enlarged view of a catheter body of the catheter in FIG. 5.

FIG. 7 is an illustration of another example of the catheter.

FIG. 8 is an enlarged view of a catheter body of the catheter in FIG. 7.

FIG. 9 is an illustration of yet another example of the catheter;

FIG. 10 is an enlarged view of a catheter body of the catheter in FIG. 9; and

FIGS. 11A-11C are a series enlarged views of example couplers configured to join a first longitudinal portion and a second longitudinal portion of a drive member.

Corresponding reference characters indicate corresponding parts throughout the drawings.

### DETAILED DESCRIPTION

The present disclosure is generally directed to a tissue-removing catheter configured to remove tissue in a body lumen. In some examples, the catheter is a rotational atherectomy device suitable for removing (e.g., abrading, cutting, excising, ablating, etc.) occlusive tissue (e.g., embolic tissue, plaque tissue, atheroma, thrombolytic tissue, stenotic tissue, hyperplastic tissue, neoplastic tissue, etc.) from a vessel wall (e.g., coronary arterial wall, etc.). The catheter may be used to facilitate percutaneous transluminal coronary angioplasty (PTCA) or the subsequent delivery of a stent. Features of the disclosed examples may also be suitable for treating chronic total occlusion (CTO) of blood vessels, and stenosis of other body lumens and other hyperplastic and neoplastic conditions in other body lumens, such as the ureter, the biliary duct, respiratory passages, the pancreatic duct, the lymphatic duct, and the like. Neoplastic cell growth may occur as a result of a tumor surrounding and intruding into a body lumen. Removal of such material can thus be beneficial to maintain patency of the body lumen.

In general, examples of the tissue-removing catheters described herein include an elongate catheter body having proximal and distal end portions, a rotatable tissue-removing element coupled to the distal end portion of the catheter body and configured to be rotated about its axis for removing tissue from the body lumen, and a handle coupled to the proximal end portion of the catheter body. The catheter body includes first and second longitudinal portions having different stiffness. In particular, the first longitudinal portion is proximal of the second longitudinal portion, and the first longitudinal portion is stiffer than the second longitudinal portion. Through this construction, the first longitudinal portion provides stiffness to the catheter body for suitable “pushability” of the catheter, and the second longitudinal portion provides flexibility to the catheter body for suitable “navigation” or “trackability” of the catheter. As used in the art, “pushability” refers to relationship between push force applied to the proximal end portion of the catheter and advancement of the catheter body in the body lumen. As used in the art, the “navigation” or “trackability” of the catheter body refers to the ability of the catheter body to move through turns or obstructions in the lumen/anatomy. The first longitudinal portion may also have a greater strength than the second longitudinal portion. Overall, and in general, the first longitudinal portion may be more robust to dynamic loading.

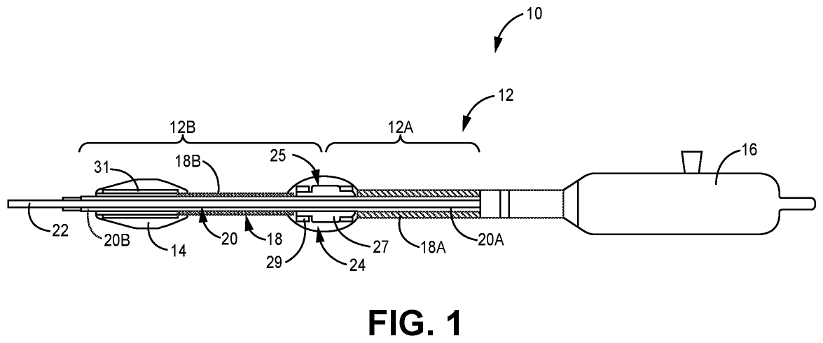

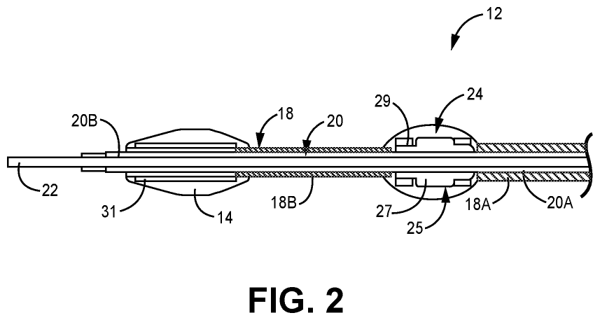

Referring to the drawings, and in particular FIGS. 1 and 2, a tissue-removing catheter is generally indicated at reference numeral 10. The illustrated catheter 10 includes an elongate catheter body, generally indicated at reference numeral 12, having proximal and distal end portions; a rotatable tissue-removing element 14 coupled to the distal end portion of the catheter body and configured to be rotated about its axis for removing tissue from the body lumen; and a handle 16 coupled to the proximal end portion of the catheter body. In one example, the catheter body 12 is sized for being received in a blood vessel of a subject. Thus, the catheter body 12 may have a maximum size of 3, 4, 5, 6, 7, 8, 9, 10, or 12 French (1, 1.3, 1.7, 2, 2.3, 2.7, 3, 3.3, or 4 mm) and may have a working length of 20, 30, 40, 60, 80, 100, 120, 150, 180 or 210 cm, depending on the body lumen. While the remaining discussion is directed toward a catheter for removing tissue in blood vessels, it will be appreciated that the teachings of the present disclosure also apply to other types of tissue-removing catheters, including, but not limited to, catheters for penetrating and/or removing tissue from a variety of occlusive, stenotic, or hyperplastic material in a variety of body lumens.

The catheter body 12 includes first and second longitudinal portions, generally indicated at 12A and 12B, respectively, having different stiffness. In particular, the first longitudinal portion 12A is proximal of the second longitudinal portion 12B, and the first longitudinal portion 12A is stiffer than the second longitudinal portion 12B. The catheter body 12 of the illustrated catheter 10 includes an elongate drive member, generally indicated at 18, and an elongate inner liner, generally indicated at 20, each of which extend along the length of the catheter body. In general, as shown best in FIG. 1, a proximal end portion of the elongate drive member 18 is operatively coupled to the handle 16, and in particular, to a drive (e.g., a motor) within the handle, and a distal end portion of the elongate drive member is operatively coupled to the tissue-removing element 14. The drive of the handle 16 rotates the elongate drive member 18 about its axis to thereby impart rotation of the tissue-removing element 14 about its axis. The inner liner 20 is received within the drive member 18 and is configured to receive a guidewire 22 therein. In one or more examples, a proximal end portion of the inner liner 20 is fixedly secured to the handle 16 such that the elongate drive member and the tissue-removing element 14 rotate relative to the inner liner. In one or more embodiment, the inner liner 20 extends through the tissue-removing element 14, as best shown in FIG. 2, and may extend distally beyond the distal end of the tissue-removing element. It is understood that in other embodiments, the catheter 10 may not include the inner liner.

In one or more embodiments, the catheter 10 includes an isolation sheath. The catheter body 12 is disposed in the isolation sheath, and the catheter body is longitudinally movable relative to and in the isolation sheath. The isolation sheath isolates the body lumen from at least a portion of the drive member 18 and inner liner 20. In one example, the isolation sheath has an inner diameter of about 0.050 inches (1.27 mm), an outer diameter of about 0.055 inches (1.4 mm), and a length of about 1500 mm (59 inches). The isolation sheath can have other dimensions without departing from the scope of the disclosure. In one example, the isolation sheath is made from Polytetrafluorethylene (PTFE). Alternatively, the isolation sheath may comprise a multi-layer construction. For example, the isolation sheath may comprise an inner layer of perfluoroalkoxy alkane (PFA), a middle braided wire layer, and an outer layer including a Pebax® (a polyether-block-amide available from Arkema, The King of Prussia, Pennsylvania).

The elongate drive member 18 has length and a longitudinal axis extending between proximal and distal ends of the drive member. In the present illustrated example, the elongate drive member 18 includes first and second longitudinal portions (e.g., proximal and distal longitudinal portions) generally indicated at 18A and 18B, respectively, having different stiffness. In particular, the first longitudinal portion 18A is proximal of the second longitudinal portion 18B, and the first longitudinal portion 18A is stiffer than the second longitudinal portion 18B. In general, the first longitudinal portion 18A of the elongate drive member 18 at least partially defines the first longitudinal portion 12A of the catheter body 12, and the second longitudinal portion 18B of the elongate drive member at least partially defines the second longitudinal portion 12B of the catheter body. In one example, the first longitudinal portion 18A of the drive member 18 extends at least about 90% of the length of the drive member 18, for example, between about 90% and 99% of the length of the drive member, and the second longitudinal portion 18B of the drive member extends from about 10% to about 1% of the length of the drive member 18.

In one example, the first longitudinal portion 18A of the drive member 18 comprises a first drive coil (e.g., stainless steel coil), and the second longitudinal portion 18B of the drive member comprises a second drive coil (e.g., stainless steel coil), as best seen in FIGS. 1 and 2. The first and second drive coils 18 are configured transfer rotation and torque from the drive (e.g., motor) to the tissue-removing element 14. Configuring the first and second longitudinal portions 18A, 18B of the drive member 18 as coiled structures allows for the rotation and torque of the drive member to be applied to the tissue-removing element 14 when the catheter 10 is traversed across a curved path. The first drive coil (of the first longitudinal portion 18A) may include one or more filars having a diameter greater than a diameter of one or more filars of the second drive coil (of the second longitudinal portion 18B) such that the first drive coil is stiffer than the second drive coil. Other ways of making the first drive coil of the first longitudinal portion 18A stiffer than the second drive coil of the second longitudinal portion 18B are possible, including but not limited to, making spacing between the filar(s) of the first drive coil less than the spacing between the filar(s) of the second drive coil, and/or laminating the first drive coil with a material that increases its stiffness, and/or making the lay angle of the filar(s) of the first drive coil greater than the lay angle of the filar(s) of the second drive coil, and/or filar groupings, among other ways. In one example, each of the first and second drive coils has an inner diameter of about 0.023 inches (0.6 mm) and an outer diameter of about 0.035 inches (0.9 mm). Each drive coil 18 may have a single layer construction. In another example, the material of the first drive coil may be stiffer than the material of the second drive coil. For example, the first drive coil may comprise a stiffer stainless steel, Nitinol (i.e., a nickel titanium alloy), or another material stiffer than the material of the second drive coil.

The presently illustrated elongate drive member 18 includes a coupler 24 coupling the first longitudinal portion 18A of the drive member to the second longitudinal portion 18B such that rotation of the first longitudinal portion 18A is imparted to the second longitudinal portion 18B. For example, as shown in FIGS. 1 and 2, a proximal end of the coupler 24 is coupled (e.g., fixedly secured) to a distal end of the first longitudinal portion 18A, and a distal end of the coupler 24 is coupled (e.g., fixedly secured) to a proximal end of the second longitudinal portion 18B. In the illustrated example, the first longitudinal portion 18A of the drive member 18 extends between the handle 16 and the coupler 24, and the second longitudinal portion 18B of the drive member extends between and couples together the coupler 24 and the tissue-removing element 14. Generally, the second longitudinal portion 18B may have a length of about 10 mm to about 50 mm along its axis (e.g., longitudinal axis). The proximal longitudinal portion 18A of the drive member 18 is designed and constructed for pushability and torque strength to help aid in directing the catheter 10 to lesions within the body lumen. The distal longitudinal portion 18B of the drive member 18 is designed and constructed for navigation by having relatively higher flexibility and reduced stiffness to reduce hang up of the catheter 10 on portions of the body lumen while being directed to the lesions within the body lumen. The inner liner 20 passes longitudinally through the coupler 24 and is coupled thereto to inhibit longitudinal movement of the inner liner relative to the drive member 18 and the tissue-removing element 14, while enabling rotation of the drive member and the tissue-removing element relative to the inner liner.

The present illustrated coupler 24 shown in FIGS. 1 and 2 comprises a coupler body fixedly connecting the first and second longitudinal portions 18A, 18B of the drive member 18, and a bearing assembly 25 within the coupler body configured to provide relative rotation between the coupler body and the inner liner 20. The coupler body may have a maximum outer diameter that is less than or equal to a maximum outer diameter of the tissue-removing element 14. The illustrated coupler body has a smooth outer surface (e.g., elliptical) and may be generally rounded to inhibit hang-ups of the coupler 24 in the body lumen. The coupler body may be formed from metal (e.g., stainless steel), polymer, ceramic, or other material.

The bearing assembly 25 of the coupler 24 includes a bushing 27 secured to the inner liner 20, and one or more bearings 29 (e.g., proximal and distal bearings) rotatably engaging the bushing. The inner liner 20 extends through the bushing 27 and may be secured thereto such as by adhesive or in other ways. In one example, the bushing 27 is made from polyetheretherketone (PEEK) and polytetrafluoroethylene (PTFE). In another example, the bushing 27 is made from polyetheretherketone (PEEK) with carbon fiber filler. The PEEK/carbon fiber bushing 27 may be preferred for its performance in high temperatures, low coefficient of friction, and wear resistance. For example, the PEEK/carbon fiber bushing 27 can maintain its structural integrity at temperatures exceeding 300° C. However, the bushing 27 can be formed from other material without departing from the scope of the disclosure. The proximal and distal bearings 29 are ring-shaped (e.g., annular) and surround proximal and distal outer surfaces of the bushing 27. In one example, the bearings 29 are made from zirconia (zirconium oxide) or other material. The proximal and distal bearings 29 are secured within recesses of the coupler body, and a center shoulder of the bushing 27 is disposed longitudinally between the proximal and distal bearings 29. The proximal and distal bearings 29 rotate with the coupler body and the drive member 18 and ride on the outer surface of the bushing 27. Through this construction, the coupler 24 inhibits longitudinal movement of the inner liner 20 relative to the drive member 18, the coupler body, and the tissue-removing element 14 while enabling rotation of the drive member and the tissue-removing element 14 relative to the inner liner 20. The coupler 24 may have other constructions configured to inhibit longitudinal movement of the inner liner 20 relative to the drive member 18 and the tissue-removing element 14 while enabling rotation of the drive member 18 and the tissue-removing element 14 relative to the inner liner 20.

In the present illustrated example, the inner liner 20 also includes first and second longitudinal portions 20A, 20B (e.g., proximal and distal longitudinal portions) having different stiffness. In particular, the first longitudinal portion 20A is proximal of the second longitudinal portion 20B, and the first longitudinal portion is stiffer than the second longitudinal portion. In this example, the first longitudinal portion 20A of the inner liner 20, in combination with the first longitudinal portion 18A of the elongate drive member 18, at least partially defines the first longitudinal portion 12A of the catheter body 12, and the second longitudinal portion 20B of the inner liner, in combination with the second longitudinal portion 18B of the elongate drive member, at least partially defines the second longitudinal portion 12B of the catheter body. In other examples, the inner liner 20 may have a uniform stiffness along its length.

In one example, material(s) of the proximal and distal longitudinal portions 20A, 20B of the inner liner 20 may be different, leading to proximal longitudinal portion of the inner liner being stiffer than the distal longitudinal portion. For example, the proximal portion 20A of the inner liner 20 may comprise or consist of an outer layer of one or more of a polyimide, a polyamide (e.g., nylon 12), a polyether block amide (e.g., PEBAX, such as PEBAX 7233); and inner layer comprising a braided metal; and an inner liner comprising one or more of fluorinated ethylene propylene, polytetrafluoroethylene, high density polyethylene, polycarbonate urethane, or polyether ether ketone. In another example, different structures of the proximal and distal longitudinal portions 20A, 20B of the inner liner 20 may enable the proximal longitudinal portion to be stiffer than the distal longitudinal portion. The inner liner 20 may be formed with the same material along its length, and the proximal longitudinal portion 20A may include a braid (e.g., metal braid) coupled to be a material to make the proximal longitudinal portion stiffer than the distal longitudinal portion 20B that does not include the braid or other structure that adds stiffness to the portion. For example, the inner liner 20 may comprise an inner PTFE layer, and an outer layer of polyimide. The PTFE inner layer provides the inner liner 20 with a lubricous interior which aids in the passing of the guidewire 22 though the inner liner. The outer polyimide layer provides wear resistance as well as having a lubricous quality which reduces friction between the inner liner 20 and the drive coil 18. Additionally, a lubricious film, such as silicone, can be added to the inner liner 20 to reduce friction between the inner liner and the drive coil 18. The proximal longitudinal portion 20A of the inner liner 20 includes braid (e.g., braided stainless steel) intermediate the inner and outer layers to provide rigidity and strength to the proximal longitudinal portion. The distal longitudinal portion 20B of the inner liner 20 may be free from the braid or have a braid with varied density which transitions from stiff to flexible to provide suitable flexibility to the distal longitudinal portion. The inner liner 20 may be of other constructions.

In one example, the inner liner 20 has an inner diameter of about 0.016 inches (0.4 mm), an outer diameter of about 0.021 inches (0.5 mm), and a length of about 59 inches (1500 mm). The inner diameter of the inner liner 20 provides clearance for the standard 0.014-inch guidewire 22. The outer diameter of the inner liner 20 provides clearance for the drive coil 18 and tissue-removing element 14. Having a space between the inner liner 20 and the drive coil 18 reduces friction between the two components as well as allows for saline perfusion between the components.

The tissue-removing element 14 engages and removes tissue in the body lumen when centered in the lesion. Any suitable tissue-removing element 14 for removing tissue in the body lumen as it is rotated may be used in one or more examples. In the illustrated example, the tissue-removing element 14 may comprise an abrasive burr configured to abrade tissue in the body lumen when the drive (e.g., motor), rotates the abrasive burr. In other examples, the tissue-removing element 14 may include one or more cutting elements having smooth or serrated cutting edges, a macerator, a thrombectomy wire, etc. In the illustrated example, a plain bearing 31 is received in and secured to the tissue-removing element 14 and is configured to engage and rotate about the inner liner 20.

As described above, the tissue-removing element 14 is coupled to (e.g., directly coupled to) the distal end of the distal longitudinal portion 18B of the drive member 18. When coupled, rotation from the drive member 18 is transferred to the tissue-removing element 14. The tissue-removing element 14 may be welded and/or crimped and/or adhered to the drive member 18. The tissue-removing element 14 may be secured to the drive member 18 in other ways. In one or more examples of catheter 10, a connector (e.g., a hypotube) may be used to secure the drive member to the tissue-removing element 14.

In one example, to remove tissue in the body lumen of a subject, a practitioner inserts the guidewire 22 into the body lumen of the subject, to a location distal of the tissue that is to be removed. Subsequently, the practitioner inserts the proximal end portion of the guidewire 22 through a guidewire lumen of the inner liner 20 and through the handle 16 so that the guidewire extends through a proximal port in the handle. With the catheter 10 loaded onto the guidewire 22, the practitioner advances the catheter along the guidewire until the tissue-removing element 14 is positioned proximal and adjacent the tissue. When the tissue-removing element 14 is positioned proximal and adjacent the tissue, the practitioner actuates the motor using an actuator to rotate the drive member 18 and the tissue-removing element 14 mounted on the drive coil. The tissue-removing element 14 abrades (or otherwise removes) the tissue in the body lumen as it rotates. While the tissue-removing element 14 is rotating, the practitioner may selectively move the drive coil 18 and inner liner 20 distally along the guidewire 22 and relative to an outer sheath to abrade the tissue and, for example, increase the size of the passage through the body lumen. The practitioner may also move the drive coil 18 and inner liner 20 proximally along the guidewire 22, and may repetitively move the components in distal and proximal directions to obtain a back-and-forth motion of the tissue-removing element 14 across the tissue by sliding an advancer. During the abrading process, the coupling allows the drive coil 18 and tissue-removing-element 14 to rotate around the inner liner 20. The inner liner 20 also isolates the guidewire 22 from the rotating drive coil 18 and tissue-removing element 14 to protect the guidewire from being damaged by the rotating components. As such, the inner liner 20 is configured to withstand the torsional and frictional effects of the rotating drive coil 18 and tissue-removing element 14 without transferring those effects to the guidewire 22. When the practitioner is finished using the catheter 10, the catheter can be withdrawn from the body lumen and unloaded from the guidewire 22 by sliding the catheter proximally along the guidewire. The guidewire 22 used for the abrading process may remain in the body lumen for use in a subsequent procedure.

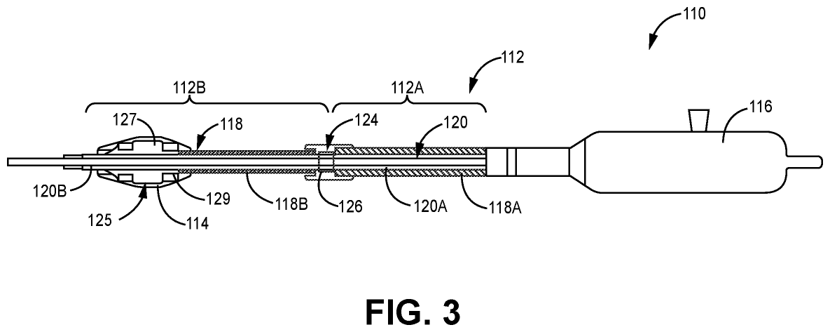

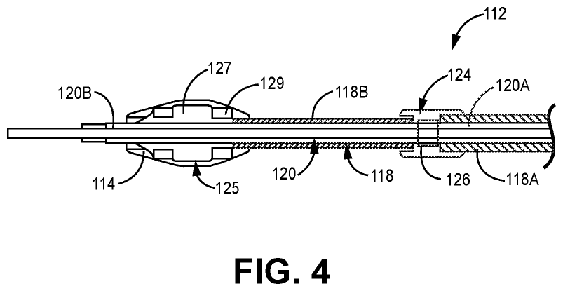

Referring to FIGS. 3 and 4, another example of a catheter is generally indicated at reference numeral 110. This catheter 110, shown in FIG. 3 is similar to the prior catheter 10, shown in FIG. 1. The catheter 110 includes a catheter body 112, a tissue-removing element 114 coupled to the distal end of the catheter body, and a handle 116 coupled to the proximal end of the catheter body. The tissue-removing element 114 and the handle 116 may be substantially similar to the tissue-removing element 14 and the handle 16 of the previous example. As with the prior catheter body 12 shown in FIGS. 1 and 2, the present catheter body 112, shown in FIGS. 3 and 4, includes first and second longitudinal portions 112A, 112B (e.g., proximal and distal longitudinal portions) having different stiffness. In particular, the first longitudinal portion 112A is proximal of the second longitudinal portion 112B, and the first longitudinal portion is stiffer than the second longitudinal portion. Moreover, the present catheter body 112 includes a drive member 118 and an inner liner 120. Respective proximal longitudinal portions 118A, 120A and distal longitudinal portions 118B, 120B of the drive member 118 and the inner liner 120 may be the same as the prior example. Accordingly, the teachings set forth above with respect to the proximal longitudinal portions 18A, 20A, and distal longitudinal portions 18B, 20B of the drive member 18 and the inner liner 20 apply to the present example. As explained below, the main difference is that the present catheter 110 includes a coupler 124 of a different construction than the prior coupler 24.

As with the prior coupler 24, the present illustrated coupler 124 couples together the proximal and distal longitudinal portions 118A, 118B of the drive member 118 such that rotation of the first longitudinal portion is imparted to the second longitudinal portion. For example, as best shown in FIG. 4, a proximal end of the coupler 124 is coupled (e.g., fixedly secured) to a distal end of the first longitudinal portion 118A, and a distal end of the coupler is coupled (e.g., fixedly secured) to a proximal end of the second longitudinal portion 118B. In the illustrated example, the first longitudinal portion 118A of the drive member 118 extends between the handle 116 and the coupler 124, and the second longitudinal portion 118B of the drive member extends between and couples together the coupler and the tissue-removing element 114. The coupler may comprise a single, one-piece body. For example, the coupler 124 may comprise a hypotube configured to mate with the proximal and distal longitudinal portions 118A, 118B of the drive member 118. A maximum outer diameter of the coupler 124 may be less than or equal to a maximum outer diameter of the tissue-removing element 114.

As illustrated, the coupler 124 does not include the bearing assembly 25. Instead, a bearing assembly 125 is disposed in the tissue-removing element 114. The bearing assembly 125 includes a bushing 127 secured to the inner liner 120, and one or more bearings (e.g., proximal and distal bearings) rotatably engaging the bushing. The constructions of the bushing 127 and bearing(s) 129 may be the same as the bushing 27 and bearing(s) 29. The inner liner 120 extends through the bushing 127 and may be secured thereto such as by adhesive or in other ways. The proximal and distal bearings 129 are ring-shaped (e.g., annular) and surround proximal and distal outer surfaces of the bushing 127. The proximal and distal bearings 129 are secured within recesses of the tissue-removing element 114, and a center shoulder of the bushing 127 is disposed longitudinally between the proximal and distal bearings. The proximal and distal bearings 129 rotate with the coupler body and the drive member 118 and ride on the outer surface of the bushing 127. Through this construction, the coupler 124 inhibits longitudinal movement of the inner liner 120 relative to the drive member 118 and the tissue-removing element 114 while enabling rotation of the drive member and the tissue-removing element relative to the inner liner. The coupler 124 may have other constructions configured to inhibit longitudinal movement of the inner liner 120 relative to the drive member 118 and the tissue-removing element 114 while enabling rotation of the drive member and the tissue-removing element relative to the inner liner. In the illustrated example, a wrap 126 may be disposed at the transition of the first and second longitudinal portions 120A, 120B of the inner liner 120 to strengthen the inner liner at the transition and protect the liner from any edges within the coil/coupler which may cause damage. It is understood that the wrap 126 may be omitted. It is also understood that the inner liner 120 may have a uniform stiffness along its length.

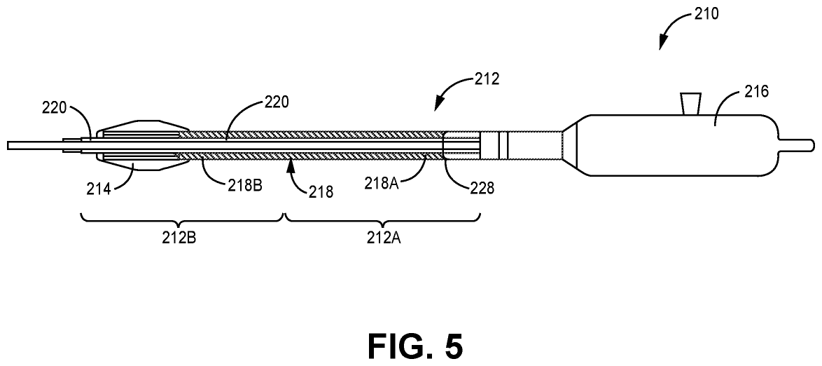

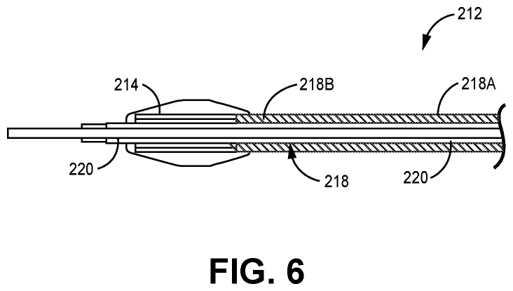

Referring to FIGS. 5 and 6, another example of a catheter is generally indicated at reference numeral 210. This catheter 210 is similar to the prior catheter 10, 110. The catheter 210 includes a catheter body 212, a tissue-removing element 214 coupled to the distal end of the catheter body, and a handle 216 coupled to the proximal end of the catheter body. The tissue-removing element 214 and the handle 216 may be substantially similar to the tissue-removing element 14, 114 and the handle 16, 116 of the previous examples. As with the prior catheter body 12, 112, the present catheter body 212 includes first and second longitudinal portions 212A, 212B having different stiffness. In particular, the first longitudinal portion 212A is proximal of the second longitudinal portion 212B, and the first longitudinal portion is stiffer than the second longitudinal portion. As explained below, the main difference is that the present catheter 210 does not include a coupler 24, 124, and a drive member 218 and an inner liner 220 are constructed differently than the previous catheter 10, 110.

The illustrated catheter 210 includes an inner liner 220 with a uniform stiffness along its length. In other examples, the inner liner 220 may have a non-uniform stiffness along its length, similar to the previous examples. The illustrated catheter 210 includes the drive member 218 that includes a drive body (e.g., a drive coil) and a laminate 228 disposed over a first longitudinal portion 218A of the drive body. The drive coil 218 has a uniform stiffness along its length; however, the laminate 228 over the first longitudinal portion 218A of the drive coil 218 increases the stiffness of the first longitudinal portion such that the stiffness of the first longitudinal portion is greater than a second longitudinal portion 218B of the drive coil that is unlaminated. This construction provides the catheter body 212 with the first (proximal) longitudinal portion 212A being stiffer than the second (distal) longitudinal portion 212B such that the first longitudinal portion provides suitable pushability and the second longitudinal portion provides suitable navigation. Suitable material for the laminate includes, but is not limited to a polyimide, a polyamide (e.g., nylon 12), a polyurethane, a polyether block amide (e.g., PEBAX, such as PEBAX 7233 or PEBAX 7033), fluorinated ethylene propylene, polytetrafluoroethylene, high density polyethylene, polycarbonate urethane, or polyether ether ketone.

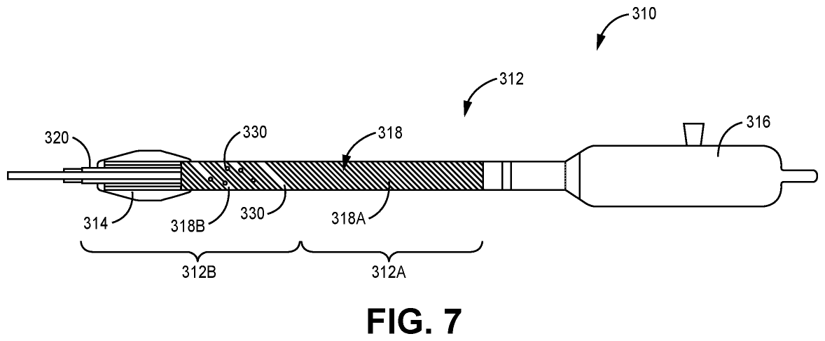

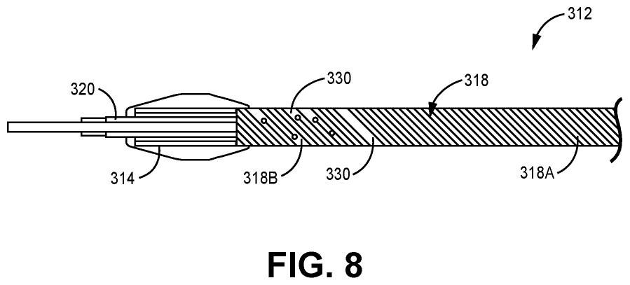

Referring to FIGS. 7 and 8, another example of a catheter is generally indicated at reference numeral 310. The main difference is that the present catheter 310 and the catheter 10, 110, 210 shown in FIGS. 1-6, the present catheter does not include a coupler 24, 124 or a laminate 228. The catheter 310 includes a catheter body 312, a tissue-removing element 314 coupled to the distal end of the catheter body, and a handle 316 coupled to the proximal end of the catheter body. The catheter 310 includes an elongate drive member, generally indicated at 318, and an elongate inner liner 320, each of which extend along the length of the catheter body 312. The tissue-removing element 314 and the handle 316 may be substantially similar to the tissue-removing element 14, 114, 214 and the handle 16, 116, 216 of the previous examples. As with the prior catheter body 12, 112, 212 the present catheter body 312 includes first and second longitudinal portions 312A, 312B having different stiffness. In particular, the first longitudinal portion 312A is proximal of the second longitudinal portion 312B, and the first longitudinal portion is stiffer than the second longitudinal portion.

The illustrated drive coil 318 includes first and second longitudinal portions generally indicated at 318A and 318B, respectively, and may have a uniform stiffness along its length; however, post-processing over the second longitudinal portion of the drive coil decreases the stiffness of the second longitudinal portion such that the stiffness of the first longitudinal portion of the drive coil that is not post processed is greater than a second proximal longitudinal portion. As shown, in one example the catheter 310, decrease in stiffness of the drive coil 318 includes post processing gaps 330 in the second longitudinal portion 318B of the drive coil. For example, but not limiting to, targeted and discrete filars can be removed to create gaps 330 in the second longitudinal portion 318B of the drive coil 318, as shown. In some examples, post processing may be imparted by, for example, but not limiting to, laser ablation, welding, or discrete stretching of the drive coil 318 in certain areas, providing a change in stiffness of the second longitudinal portion of the coil. This construction provides the catheter body 312 with the first (proximal) longitudinal portion 312A being stiffer than the second (distal) longitudinal portion 312B such that the first longitudinal portion provides suitable pushability and the second longitudinal portion provides suitable navigation.

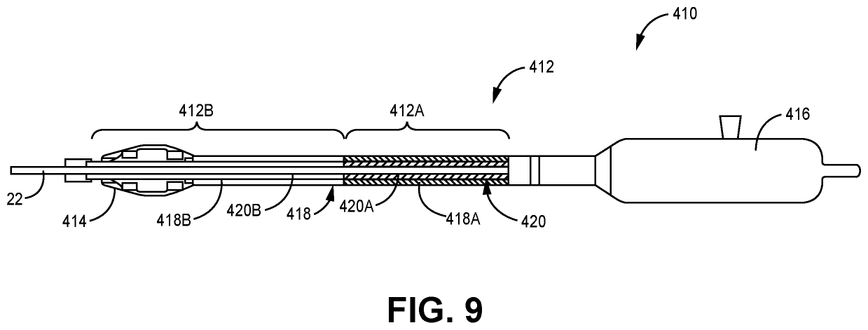

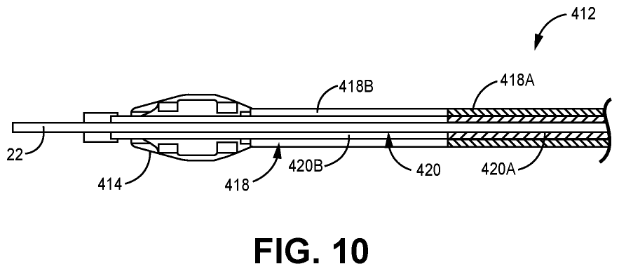

Referring to FIGS. 9 and 10, another example of a catheter is generally indicated at reference numeral 410. The main difference is that the present catheter 410 and the catheter 10, 110, 210 shown in FIGS. 1-6, the present catheter does not include a coupler 24, 124 or a laminate 228. The catheter 410 includes a catheter body 412, a tissue-removing element 414 coupled to the distal end of the catheter body, and a handle 316 coupled to the proximal end of the catheter body. The catheter 410 includes an elongate drive member, generally indicated at 418, and an elongate inner liner 420, each of which extend along the length of the catheter body 412. The tissue-removing element 414 and the handle 416 may be substantially similar to the tissue-removing element 14, 114, 214, 314 and the handle 16, 116, 216, 316 of the previous examples. As with the prior catheter body 12, 112, 212, 312 the present catheter body 412 includes first and second longitudinal portions 412A, 412B having different stiffness. In particular, the first longitudinal portion 412A is proximal of the second longitudinal portion 412B, and the first longitudinal portion is stiffer than the second longitudinal portion.

The illustrated drive coil 418 includes first and second longitudinal portions generally indicated at 418A and 418B, respectively, of different stiffness. The illustrated inner liner 420 includes first and second longitudinal portions generally indicated at 420A and 420B, respectively, of different stiffness. For example, as shown, a proximal end of the second longitudinal portions 418B, 420B of the drive coil 418 and the inner liner 420 are directly coupled to a distal end of the first longitudinal portion 418A, 420B of the drive coil and the inner liner by, for example but not limiting to welding (e.g., butt welding), crimping, and/or adhering.

In this example, the first longitudinal portion 420A of the inner liner 420, in combination with the first longitudinal portion 418A of the elongate drive member 418, at least partially defines the first longitudinal portion 412A of the catheter body 412, and the second longitudinal portion 420B of the inner liner, in combination with the second longitudinal portion 418B of the elongate drive member, at least partially defines the second longitudinal portion 412B of the catheter body. This construction provides the catheter body 412 with the first (proximal) longitudinal portion 412A being stiffer than the second (distal) longitudinal portion 412B such that the first longitudinal portion provides suitable pushability and the second longitudinal portion provides suitable navigation.

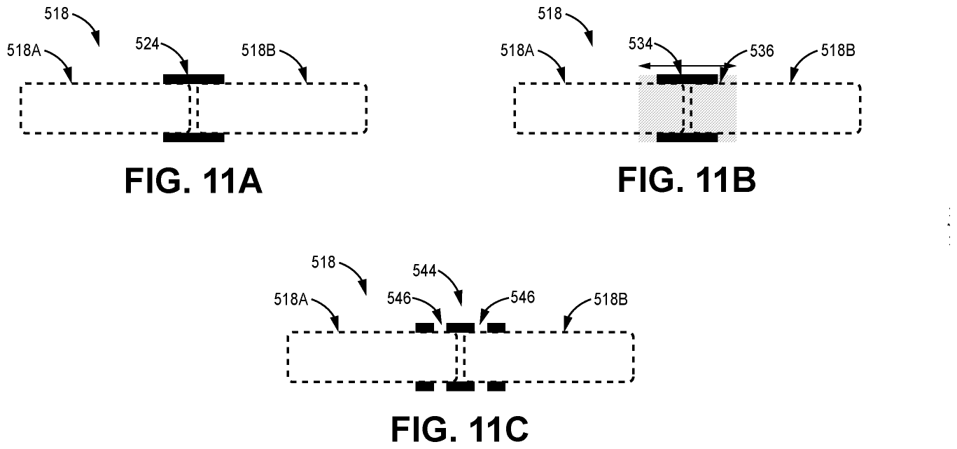

The couplers 24 and 124 described with reference to FIGS. 1-4 may, in some examples, have other configurations. FIGS. 11A-11C are a series enlarged views of example couplers configured to join a first longitudinal portion and a second longitudinal portion of a drive member. FIG. 11A illustrates a first longitudinal portion 518A of a drive member 518 joined to a second longitudinal portion 518B of the drive member 518 using a coupler 524. In the example shown in FIG. 11A, the coupler 524 is formed from a metal tube (e.g., an alloy) and is welded to a distal end portion of the first longitudinal portion 518A and a proximal end portion of the second longitudinal portion 518B. Although any suitable type of joining technique (e.g., welding, adhesive, soldering, mechanical interference, friction fit, or the like) may be used to weld the coupler 524 to the first longitudinal portion 518A and the second longitudinal portion 518B, in some implementations, a lap weld is used. The coupler 524 may have any suitable length, inner diameter, outer diameter, and wall thickness. The length, inner diameter, outer diameter, and wall thickness may be selected based at least in part on the operating conditions to which the coupler 524 will be exposed during use. In some examples, the length of the coupler 524 may be between about 1.35 mm and about 4 mm, such as between about 3.5 mm and about 4.0 mm. The wall thickness range may be between about 203 micrometer and about 406 micrometer, such as between about 254 micrometer and about 356 micrometer.

FIG. 11B illustrates another example arrangement of a first longitudinal portion 518A of a drive member 518 joined to a second longitudinal portion 518B of the drive member 518 using a coupler 534. The example shown in FIG. 11B may be similar to or substantially the same as the example shown in FIG. 11A, aside from the differences described herein. For instance, the example shown in FIG. 11B additionally includes a laminate strain relief 536 positioned around the coupler 534. The laminate strain relief 536 may include a polymer or multilayer polymer film, and may be shrunk (e.g., heat shrunk) about the coupler 534. The laminate strain relief 536 may extend proximally and distally beyond the ends of the coupler 534, and may reduce strain on the first longitudinal portion 518A and the second longitudinal portion 518B at the ends of the coupler 534. Additionally or alternatively, the laminate strain relief 536 may smooth the transition between the coupler 534 and the first longitudinal portion 518A and the transition between the coupler 534 and the second longitudinal portion 518B.

FIG. 11C illustrates an additional example arrangement of a first longitudinal portion 518A of a drive member 518 joined to a second longitudinal portion 518B of the drive member 518 using a coupler 544. The example shown in FIG. 11C may be similar to or substantially the same as the example shown in FIG. 11A, aside from the differences described herein. Unlike the coupler 524 in FIG. 11A, the coupler 544 in FIG. 11C includes weld orifices 546, which provide access points for the energy source to access the interface between the first longitudinal portion 518A and the coupler 544 and the interface between the second longitudinal portion 518B and the coupler 544. This may facilitate welding of the coupler 544 to the first longitudinal portion 518A and the second longitudinal portion 518B.

When introducing elements of the present invention or the one or more example(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above apparatuses, systems, and methods without departing from the scope of the invention, it is intended that all matter contained in the above description and shown in the accompanying drawings shall be interpreted as illustrative and not in a limiting sense.

1. A tissue-removing catheter for removing tissue in a body lumen, the tissue-removing catheter comprising:
an elongate drive member having proximal and distal ends and a length and longitudinal axis extending between the proximal and distal ends, the elongate drive member being sized and shaped to be received in the body lumen and configured to be rotated about its longitudinal axis; and
a tissue-removing element operatively coupled to the distal end of the elongate drive member, the tissue-removing element being configured to be rotated by the elongate drive member to remove the tissue in the body lumen,
wherein the elongate drive member includes a first and second longitudinal portions, the first longitudinal portion being proximal of and stiffer than the second longitudinal portion.

2. The tissue-removing catheter as set forth in claim 1, further comprising a coupler coupling together the first and second longitudinal portions of the elongate drive member.

3. The tissue-removing catheter as set forth in claim 2, wherein the coupler comprises a bearing assembly.

4. The tissue-removing catheter as set forth in claim 3, wherein the coupler includes a coupler body, and the bearing assembly is received in the coupler body.

5. The tissue-removing catheter as set forth in claim 4, wherein the bearing assembly includes a bushing and a bearing, wherein the bearing is configured to rotate about and ride on the bushing, the inner liner being secured to the bushing, the bearing being secured to the coupler body, whereby the elongate drive member and the bearing are rotatable about the inner liner and the bushing.

6. The tissue-removing catheter as set forth claim 5, wherein the coupler comprises a metal tube welded to a proximal end portion of the second longitudinal portion and a distal end portion of the first longitudinal portion.

7. The tissue-removing catheter as set forth in claim 6, wherein the first longitudinal portion of the elongate drive member extends along a majority of the length of the elongate drive member.

8. The tissue-removing catheter as set forth in claim 7, wherein the first longitudinal portion of the elongate drive member extends along at least 90% of the length of the elongate drive member.

9. The tissue-removing tissue removing catheter as set forth in claim 8, further comprising an inner liner received in and extending along the length of the elongate drive member, wherein the inner liner defines a longitudinal passage configured to receive a guidewire therein, the elongate member being rotatable about the inner liner.

10. The tissue-removing catheter as set forth in claim 9, wherein the inner liner includes first and second longitudinal portions, the first longitudinal portion being proximal of and stiffer than the second longitudinal portion.

11. The tissue-removing catheter as set forth in claim 1, wherein the first and second longitudinal portions of the elongate drive member are secured directly to one another.

12. The tissue-removing catheter as set forth in claim 11, wherein the elongate drive member includes a first drive coil defining the first longitudinal portion of the elongate drive member, and a second drive coil defining the second longitudinal portion of the elongate drive member.

13. The tissue-removing catheter as set forth in claim 12, wherein the first longitudinal portion of the elongate drive member is laminated to increase stiffness of the first longitudinal portion.

14. The tissue-removing catheter as set forth in claim 12, wherein a filar of the first drive coil comprises a diameter greater than a diameter of a filar of the second drive coil.

15. The tissue-removing catheter as set forth in claim 12, wherein at least a portion of a filar of the second drive coil is removed to increase flexibility of the second drive coil.

16. The tissue-removing catheter as set forth in claim 2, wherein the coupler comprises a metal tube welded to a proximal end portion of the second longitudinal portion and a distal end portion of the first longitudinal portion.

17. The tissue-removing catheter as set forth in claim 1, wherein the first longitudinal portion of the elongate drive member extends along a majority of the length of the elongate drive member.

18. The tissue-removing catheter as set forth in claim 17, wherein the first longitudinal portion of the elongate drive member extends along at least 90% of the length of the elongate drive member.

19. The tissue-removing catheter as set forth in claim 1, further comprising an inner liner received in and extending along the length of the elongate drive member, wherein the inner liner defines a longitudinal passage configured to receive a guidewire therein, the elongate member being rotatable about the inner liner.

20. The tissue-removing catheter as set forth in claim 19, wherein the inner liner includes first and second longitudinal portions, the first longitudinal portion being proximal of and stiffer than the second longitudinal portion.
